(19) United States
(12) Patent Application Publication
(10) Pub. No.: US 2009/0131514 A1
(43) Pub. Date: May 21, 2009

(54) BUCCAL, POLAR AND NON-POLAR SPRAYS CONTAINING PROPOFOL

(76) Inventor: Harry A. Dugger, III, Flemington, NJ (US)

Correspondence Address: DICKSTEIN SHAPIRO LLP, 1825 EYE STREET NW, Washington, DC, 20006-5403, US

(21) Appl. No.: 12/350,915

(22) Filed: Jan. 8, 2009

Related U.S. Application Data
(63) Continuation of application No. 11/442,137, filed on May 30, 2006, now abandoned.
Application No. 11/442,137 is a division of application No. 10/834,815, filed on Apr. 27, 2004.
Application No. 10/834,815 is a continuation-in-part of application No. 10/230,060, filed on Aug. 29, 2002, now abandoned.
Application No. 10/230,060 is a continuation-in-part of application No. 09/537,118, filed on Mar. 29, 2000.
Application No. 09/537,118 is a continuation-in-part of application No. PCT/US97/17899, filed on Oct. 1, 1997.

(51) Int. Cl. A61K 31/355; A61K 31/05; A61K 31/375

(52) U.S. Cl. 514/458; 514/731; 514/474

(57) ABSTRACT

Buccal aerosol sprays using polar and/or non-polar solvents have now been developed which provide propofol for rapid absorption through the oral mucosa, resulting in fast onset of effect. The buccal polar compositions of the invention comprise formulation I: propofol, a polar solvent and an optional flavoring agent; formulation II: propofol, a polar solvent, a propellant, and an optionally flavoring agent; formulation III: propofol, a non-polar solvent, and an optional flavoring agent; formulation IV: propofol, a non-polar solvent, a propellant, and an optional flavoring agent; formulation V: propofol, a mixture of a polar solvent and a non-polar solvent, and an optional flavoring agent; and formulation VI: propofol, a mixture of a polar solvent and a non-polar solvent, a propellant, and an optional flavoring agent.

### CROSS REFERENCE TO RELATED APPLICATIONS

This application is a continuation-in-part of application Ser. No. 10/230,060, filed Aug. 29, 2002, which is a continuation-in-part of application Ser. No. 09/537,118, filed Mar. 29, 2000 which is a continuation-in-part of the U.S. national phase designation of PCT/US97/17899 filed Oct. 1, 1997, the disclosures of which are incorporated by reference herein in their entirety.

### BACKGROUND OF THE INVENTION

It is known that certain biologically active compounds are better absorbed through the oral mucosa than through other routes of administration, such as through the stomach or intestine. However, formulations suitable for such administration by these latter routes present their own problems. For example, the biologically active compound must be compatible with the other components of the composition such as propellants, solvents, etc. Many such formulations have been proposed. For example, U.S. Pat. No. 4,689,233, Dvorsky et al., describes a soft gelatin capsule for the administration of the anti-coronary drug nifedipine dissolved in a mixture of polyether alcohols. U.S. Pat. No. 4,755,389, Jones et al., describes a hard gelatin chewable capsule containing nifedipine. A chewable gelatin capsule containing a solution or dispersion of a drug is described in U.S. Pat. No. 4,935,243, Borkan et al. U.S. Pat. No. 4,919,919, Aouda et al., and U.S. Pat. No. 5,370,862, Klokkers-Bethke, describe a nitroglycerin spray for administration to the oral mucosa comprising nitroglycerin, ethanol, and other components. An orally administered pump spray is described by Cholcha in U.S. Pat. No. 5,186,925. Aerosol compositions containing a hydrocarbon propellant and a drug for administration to a mucosal surface are described in U.K. 2,082,457, Su; U.S. Pat. No. 3,155,574, Silson et al., U.S. Pat. No. 5,011,678, Wang et al., and by Parnell in U.S. Pat. No. 5,128,132. It should be noted that these references discuss bioavailability of solutions by inhalation rather than through the membranes to which they are administered.

### SUMMARY OF THE INVENTION

A buccal aerosol spray or soft bite gelatin capsule using a polar or non-polar solvent has now been developed which provides biologically active compounds for rapid absorption through the oral mucosa, resulting in fast onset of effect.

The buccal aerosol spray compositions of the present invention, for transmucosal administration of a pharmacologically active compound soluble in a pharmacologically acceptable non-polar solvent comprise in weight % of total composition: pharmaceutically acceptable propellant 5-80%, nonpolar solvent 19-85%, active compound 0.05-50%, suitably additionally comprising, by weight of total composition a flavoring agent 0.01-10%. Preferably the composition comprises: propellant 10-70%, non-polar solvent 25-89.9%, active compound 0.01-40%, flavoring agent 1-8%; most suitably propellant 20-70%, non-polar solvent 25-74.75%, active compound 0.25-35%, flavoring agent 2-7.5%.

The buccal polar aerosol spray compositions of the present invention, for transmucosal administration of a pharmacologically active compound soluble in a pharmacologically acceptable polar solvent are also administrable in aerosol form driven by a propellant. In this case, the composition comprises in weight % of total composition: aqueous polar solvent 10-97%, active compound 0.1-25%, suitably additionally comprising, by weight of total composition a flavoring agent 0.05-10% and propellant: 2-10%.

Preferably the composition comprises: polar solvent 20-97%, active compound 0.1-15%, flavoring agent 0.1-5% and propellant 2-5%; most suitably polar solvent 25-97%, active compound 0.2-25%, flavoring agent 0.1-2.5% and propellant 2-4%.

The buccal pump spray composition of the present invention, i.e., the propellant free composition, for transmucosal administration of a pharmacologically active compound wherein said active compound is soluble in a pharmacologically acceptable non-polar solvent comprises in weight % of total composition: non-polar solvent 30-99.69%, active compound 0.005-55%, and suitably additionally, flavoring agent 0.1-10%.

The buccal polar pump spray compositions of the present invention, i.e., the propellant free composition, for transmucosal administration of a pharmacologically active compound soluble in a pharmacologically acceptable polar solvent comprises in weight % of total composition: aqueous polar solvent 30-99.69%, active compound 0.001-60%, suitably additionally comprising, by weight of total composition a flavoring agent 0.1-10%. Preferably the composition comprises: polar solvent 37-98.58%, active compound 0.005-55%, flavoring agent 0.5-8%; most suitably polar solvent 60.9-97.06%, active compound 0.01-40%, flavoring agent 0.75-7.5%.

In one embodiment the buccal polar pump spray composition of the present invention, i.e., the propellant free composition, for transmucosal administration of propofol comprises propofol in an amount of between about 0.1 and about 99.8 percent by weight of the total composition; and a polar solvent in an amount between about 0.05 and about 98.7 percent by weight of the total composition. Optionally, the composition can contain a taste mask and/or flavoring agent in an amount of between about 0.01 and about 5 percent by weight of the total composition. Preferably, the composition comprises propofol is present in an amount between about 1 and about 95 percent by weight of the total composition, polar solvent is present in an amount between about 1 and about 75 percent by weight of the total composition, and taste mask and/or flavoring agent is present in an amount between about 0.5 and about 4 percent by weight of the total composition. Most preferably, in the composition propofol is present in an amount between about 5 and about 90 percent by weight of the total composition, the polar solvent is present in an amount between about 5 and about 60 percent by weight of the total composition, and the taste mask and/or flavoring agent is present in an amount between about 1 and about 2 percent by weight of the total composition.

In one embodiment the propellant buccal polar pump spray composition of the present invention for the administration of propofol comprises propofol in an amount of between about 1 and about 85 percent by weight of the total composition; a polar solvent in an amount between about 1 and about 85 percent by weight of the total composition; and a propellant in an amount between about 10 and about 90 percent by weight of the total composition, wherein said propellant comprises a C₃ to C₈ hydrocarbon of linear or branched configuration. Optionally this composition comprises a taste mask and/or flavoring agent in an amount between about 0.01 and about 5 percent by weight of the total composition. Preferably, the composition comprises propofol present in an amount between about 5 and about 75 percent by weight of the total composition, the polar solvent is present in an amount between about 5 and about 75 percent by weight of the total composition, the propellant is present in an amount between about 10 and about 85 percent by weight of the composition, and the taste mask and/or flavoring agent is present in an amount between about 0.5 and about 4 percent by weight of the total composition. Most preferably, the composition comprises propofol is present in an amount between about 10 and about 70 percent by weight of the total composition, the polar solvent is present in an amount between about 10 and about 60 percent by weight of the total composition, the propellant is present in an amount between about 15 and about 65 percent by weight of the composition, and taste mask and/or flavoring agent is present in an amount between about 1 and about 2 percent by weight of the total composition.

In one embodiment a buccal non-polar pump spray composition of the present invention, i.e., the propellant free composition, for transmucosal administration of propofol comprises propofol in an amount between about 0.1 and about 99.8 percent by weight of the total composition; and a non-polar solvent in an amount between about 0.05 and about 98.7 percent by weight of the total composition. Optionally, composition comprises a taste mask and/or flavoring agent in an amount between about 0.01 and about 5 percent by weight of the total composition. Preferably, the propofol is present in an amount between about 1 and about 95 percent by weight of the total composition; the non-polar solvent is present in an amount between about 1 and about 75 percent by weight of the total composition; and the taste mask and/or flavoring agent is present in an amount between about 0.5 and about 4 percent by weight of the total composition. Most preferably, the propofol is present in an amount between about 5 and about 90 percent by weight of the total composition; the non-polar solvent is present in an amount between about 5 and about 60 percent by weight of the total composition; and the taste mask and/or flavoring agent is present in an amount between about 1 and about 2 percent by weight of the total composition.

In one embodiment the propellant buccal non-polar pump spray composition of the present invention for the administration of propofol comprises propofol in an amount between about 1 and about 85 percent by weight of the total composition; a non-polar solvent in an amount between about 1 and about 85 percent by weight of the total composition; and a propellant in an amount between about 10 and about 90 percent by weight of the total composition, wherein said propellant comprises a C₃ to C₈ hydrocarbon of linear or branched configuration. Optionally, the composition comprises a taste mask and/or flavoring agent in an amount of between about 0.01 and about 5 percent by weight of the total composition. Preferably, the propofol is present in an amount between about 5 and about 75 percent by weight of the total composition; the non-polar solvent is present in an amount between about 5 and about 75 percent by weight of the total composition; the propellant is present in an amount between about 10 to about 85 percent by weight of the total composition; and the taste mask and/or flavoring agent is present in an amount between about 0.5 to about 4 percent by weight of the total composition. Most preferably, the propofol is present in an amount between about 10 and about 70 percent by weight of the total composition; the non-polar solvent is present in an amount between about 10 and about 60 percent by weight of the total composition; the propellant is present in an amount between about 15 to about 65 percent by weight of the total composition; and the taste mask and/or flavoring agent is present in an amount between about 1 to about 2 percent by weight of the total composition.

In one embodiment a buccal pump spray composition of the present invention, i.e., the propellant free composition, for transmucosal administration of propofol comprises propofol in an amount of between about 0.1 and about 99.8 percent by weight of the total composition; a polar solvent in an amount of between about 0.05 to about 98.7 percent by weight of the total composition; and a non-polar solvent in an amount of between about 0.1 to about 80 percent by weight of the total composition, wherein the ratio of the polar solvent to the non-polar solvent can range from about 1:99 to about 99:1. Optionally, the composition comprises a taste mask and/or flavoring agent in an amount of between about 0.01 and about 5 percent by weight of the total composition. Preferably, the propofol is present in an amount between about 1 to about 95 percent by weight of the total composition, the polar solvent is present in an amount between about 1 to about 75 percent by weight of the total composition, the non-polar solvent is present in an amount between about 0.5 to about 75 percent by weight of the total composition, and the taste mask and/or flavoring agent is present in an amount between about 0.5 to about 4 percent by weight of the total composition. Most preferably, the propofol is present in an amount between about 5 to about 90 percent by weight of the total composition, the polar solvent is present in an amount between about 5 to about 60 percent by weight of the total composition, the non-polar solvent is present in an amount between about 1 to about 60 percent by weight of the total composition, and the taste mask and/or flavoring agent is present in an amount between about 1 to about 2 percent by weight of the total composition.

In one embodiment the propellant buccal pump spray composition of the present invention for the administration of propofol comprises propofol in an amount between about 1 and about 80 percent by weight of the total composition; a polar solvent in an amount of between about 2 to about 80 percent by weight of the total composition; a non-polar solvent in an amount of between about 1 to about 80 percent by weight of the total composition, wherein the ratio of the polar solvent to the non-polar solvent can range from about 1:99 to about 99:1; and a propellant in an amount between about 10 to about 90 percent by weight of the total composition, wherein said propellant comprises a C₃ to C₈ hydrocarbon of linear or branched configuration. Optionally, the composition comprises a taste mask and/or flavoring agent is present in an amount between about 0.01 and about 5 percent by weight of the total composition. Preferably, the propofol is present in an amount from between about 5 to about 75 percent by weight of the total composition, the polar solvent is present in an amount between about 5 to about 75 percent by weight of the total composition, the non-polar solvent is present in an amount between about 2 to about 75 percent by weight of the total composition, the propellant is present in an amount between about 10 to about 85 percent by weight of the total composition, and the taste mask and/or flavoring agent is present in an amount between about 0.5 and about 4 percent by weight of the total composition. Most, preferably, the propofol is present in an amount from between about 10 to about 60 percent by weight of the total composition, the polar solvent is present in an amount between about 10 to about 60 percent by weight of the total composition, the non-polar solvent is present in an amount between about 10 to about 60 percent by weight of the total composition, the propellant is present in an amount between about 15 to about 65 percent by weight of the total composition, and the taste mask and/or flavoring agent is present in an amount between about 1 and about 2 percent by weight of the total composition.

The soft bite gelatin capsules of the present invention for transmucosal administration of a pharmacologically active compound, at least partially soluble in a pharmacologically acceptable non-polar solvent, having charged thereto a fill composition comprise in weight % of total composition: non-polar solvent 4-99.99%, emulsifier 0-20%, active compound 0.01-80%, provided that said fill composition contains less than 10% of water, suitably additionally comprising, by weight of the composition: flavoring agent 0.01-10%. Preferably, the soft bite gelatin capsule comprises: non-polar solvent 21.5-99.975%, emulsifier 0-15%, active compound 0.025-70%, flavoring agent 1-8%; most suitably: nonpolar solvent 28.5-97.9%, emulsifier 0-10%, active compound 0.1-65.0%, flavoring agent 2-6%.

The soft bite polar gelatin capsules of the present invention for transmucosal administration of a pharmacologically active compound, at least partially soluble in a pharmacologically acceptable polar solvent, having charged thereto a composition comprising in weight % of total composition: polar solvent 25-99.89%, emulsifier 0-20%, active compound 0.01-65%, provided that said composition contains less than 10% of water, suitably additionally comprising, by weight of the composition: flavoring agent 01-10%. Preferably, the soft bite gelatin capsule comprises: polar solvent 37-99.95%, emulsifier 0-15%, active compound 0.025-55%, flavoring agent 1-8%; most suitably: polar solvent 44-96.925%, emulsifier 0-10%, active compound 0.075-50%, flavoring agent 2-6%.

It is an object of the invention to coat the mucosal membranes either with extremely fine droplets of spray containing the active compounds or a solution or paste thereof from bite capsules.

It is also an object of the invention to administer to the oral mucosa of a mammalian in need of same, preferably man, by spray or bite capsule, a predetermined amount of a biologically active compound by this method or from a soft gelatin capsule.

A further object is a sealed aerosol spray container containing a composition of the non polar or polar aerosol spray formulation, and a metered valve suitable for releasing from said container a predetermined amount of said composition.

As the propellant evaporates after activation of the aerosol valve, a mist of fine droplets is formed which contains solvent and active compound.

The propellant is a non-Freon material, preferably a C₃₋₈ hydrocarbon of a linear or branched configuration. The propellant should be substantially non-aqueous. The propellant produces a pressure in the aerosol container such that under expected normal usage it will produce sufficient pressure to expel the solvent from the container when the valve is activated but not excessive pressure such as to damage the container or valve seals.

The non-polar solvent is a non-polar hydrocarbon, preferably a C₇₋₁₈ hydrocarbon of a linear or branched configuration, fatty acid esters, and triglycerides, such as miglyol. The solvent must dissolve the active compound and be miscible with the propellant, i.e., solvent and propellant must form a single phase at a temperature of 0-40° C. a pressure range of between 1-3 atm.

The polar and non-polar aerosol spray compositions of the invention are intended to be administered from a sealed, pressurized container. Unlike a pump spray, which allows the entry of air into the container after every activation, the aerosol container of the invention is sealed at the time of manufacture. The contents of the container are released by activation of a metered valve, which does not allow entry of atmospheric gasses with each activation. Such containers are commercially available.

A further object is a pump spray container containing a composition of the pump spray formulation, and a metered valve suitable for releasing from said container a predetermined amount of said composition.

A further object is a soft gelatin bite capsule containing a composition of as set forth above. The formulation may be in the form of a viscous solution or paste containing the active compounds. Although solutions are preferred, paste fills may also be used where the active compound is not soluble or only partially soluble in the solvent of choice. Where water is used to form part of the paste composition, it should not exceed 10% thereof. (All percentages herein are by weight unless otherwise indicated.)

The polar or non-polar solvent is chosen such that it is compatible with the gelatin shell and the active compound. The solvent preferably dissolves the active compound. However, other components wherein the active compound is not soluble or only slightly soluble may be used and will form a paste fill.

Soft gelatin capsules are well known in the art. See, for example, U.S. Pat. No. 4,935,243, Borkan et al., for its teaching of such capsules. The capsules of the present invention are intended to be bitten into to release the low viscosity solution or paste therein, which will then coat the buccal mucosa with the active compounds. Typical capsules, which are swallowed whole or bitten and then swallowed, deliver the active compounds to the stomach, which results in significant lag time before maximum blood levels can be achieved or subject the compound to a large first pass effect. Because of the enhanced absorption of the compounds through the oral mucosa and no chance of a first pass effect, use of the bite capsules of the invention will eliminate much of the lag time, resulting in hastened onset of biological effect. The shell of a soft gelatin capsule of the invention may comprise, for example: gelatin: 50-75%, glycerin 20-30%, colorants 0.5-1.5%, water 5-10%, and sorbitol 2-10%.

The active compound may include, biologically active peptides, central nervous system active amines, sulfonyl ureas, antibiotics, antifungals, antivirals, sleep inducers, antiasthmatics, bronchial dilators, antiemetics, histamine H-2 receptor antagonists, barbiturates, prostaglandins and neutraceuticals.

The active compounds may also include antihistamines, alkaloids, hormones, benzodiazepines and narcotic analgesics. While not limited thereto, these active compounds are particularly suitable for non-polar pump spray formulation and application.

The active compounds may also include p-FOX (fatty acid oxidation) inhibitors, acetylcholinesterase inhibitors, nerve impulse inhibitors, anti-cholinergics, anti-convulsants, anti-psychotics, anxiolytic agents, dopamine metabolism inhibitors, agents to treat post stroke sequelae, neuroprotectants, agents to treat Alzheimer's disease, neurotransmitters, neurotransmitter agonists, sedatives, agents for treating attention deficit disorder, agents for treating narcolepsy, central adregenic antagonists, anti-depression agents, agents for treating Parkinson's disease, benzodiazepine antagonists, stimulants, neurotransmitter antagonists, tranquilizers, or a mixture thereof.

### BRIEF DESCRIPTION OF THE DRAWING

FIG. 1. is a schematic diagram showing routes of absorption and processing of pharmacologically active substances in a mammalian system.

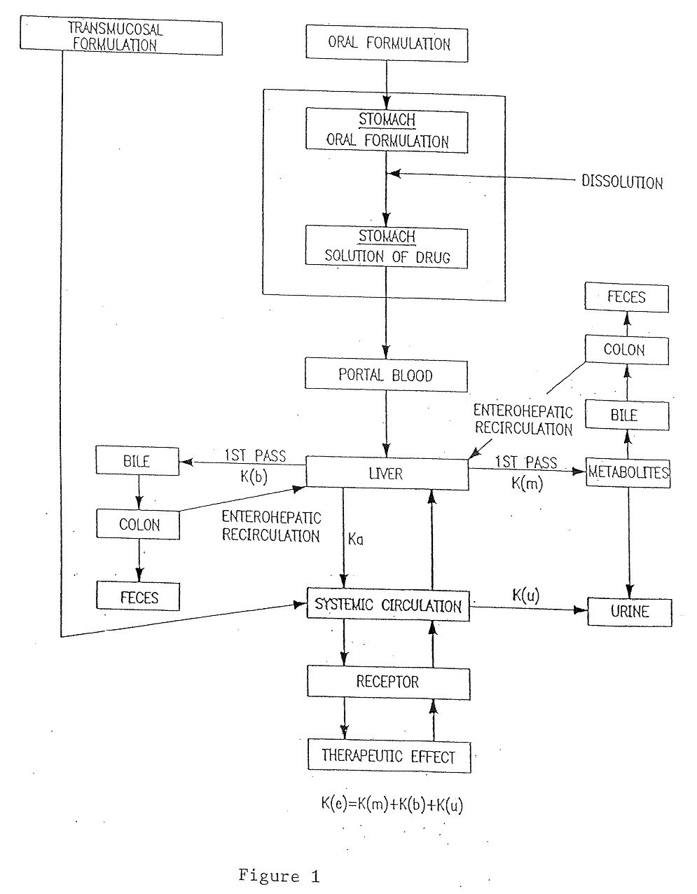

### DESCRIPTION OF THE PREFERRED EMBODIMENTS

The preferred active compounds of the present invention are in an ionized, salt form or as the flee base of the pharmaceutically acceptable salts thereof (provided, for the aerosol or pump spray compositions, they are soluble in the spray solvent). These compounds are soluble in the non-polar solvents of the invention at useful concentrations or can be prepared as pastes at useful concentrations. These concentrations may be less than the standard accepted dose for these compounds since there is enhanced absorption of the compounds through the oral mucosa. This aspect of the invention is especially important when there is a large (40-99.99%) first pass effect.

As propellants for the non polar sprays, propane, N-butane, iso-butane, N-pentane, iso-pentane, and neo-pentane, and mixtures thereof may be used. N-butane and iso-butane, as single gases, are the preferred propellants. It is permissible for the propellant to have a water content of no more than 0.2%, typically 0.1-0.2%. All percentages herein are by weight unless otherwise indicated. It is also preferable that the propellant be synthetically produced to minimize the presence of contaminants which are harmful to the active compounds. These contaminants include oxidizing agents, reducing agents, Lewis acids or bases, and water. The concentration of each of these should be less than 0.1%, except that water may be as high as 0.2%.

Suitable non-polar solvents for the capsules and the non-polar sprays include (C₂-C₂₄) fatty acid (C₂-C₆) esters, C₇-C₁₈ hydrocarbon (of linear or branched configuration), C₂-C₆ alkanoyl esters, and the triglycerides of the corresponding acids, e.g. C₂-C₆ carboxylic acids. When the capsule fill is a paste, other liquid components may be used instead of the above low molecular weight solvents. These include soya oil, corn oil, other vegetable oils.

As solvents for the polar capsules or sprays there may be used low molecular weight polyethyleneglycols (PEG) of 400-1000 Mw (preferably 400-600), low molecular weight (C₂-C₈) mono and polyols and alcohols of C₇-C₁₈ linear or branch chain hydrocarbons, glycerin may also be present and water may also be used in the sprays, but only in limited amount in the capsules.

It is expected that some glycerin and water used to make the gelatin shell will migrate from the shell to the fill during the curing of the shell. Likewise, there may be some migration of components from the fill to the shell during curing and even throughout the shelf-life of the capsule.

Therefore, the values given herein are for the compositions as prepared, it being within the scope of the invention that minor variations will occur.

The preferred flavoring agents are synthetic or natural oil of peppermint, oil of spearmint, citrus oil, fruit flavors, sweeteners (sugars, aspartame, saccharin, etc.), and combinations thereof.

The compositions may further include a taste mask. The term “taste mask” as used herein means an agent that can hide or minimize an undesirable flavor such as a bitter or sour flavor. A representative taste masks is a combination of vanillin, ethyl vanillin, maltol, iso-amyl acetate, ethyl oxyhydrate, anisic aldehyde, and propylene glycol (commercially available as “PFC 9885 Bitter Mask” from Pharmaceutical Flavor Clinic of Camden, N.J.). A taste mask in combination with a flavoring agent is particularly advantageous when the active compound is an alkaloid since alkaloids often have a bitter taste.

The compositions of the invention may also include additional components such as absorption enhancers and antioxidants. Examples of suitable absorption enhancers include without limitation oleic acid, 23-lauryl ether, aprotinin, azone, benzalkonium chloride, cetylpyridinium chloride, cetyltrimethylammonium bromide, cyclodextrin, dextran sulfate, lauric acid, lauric acid/propylene glycol, lysophosphatidylcholine, menthol, methoxysalicylate, methyloleate, phosphatidylcholine, polyoxyethylene, polysorbate 80, sodium EDTA (ethylenediamine tetraacetic acid), sodium glycocholate, sodium glycodeoxycholate, sodium lauryl sulfate, sodium salicylate, sodium taurocholate, sodium taurodeoxycholate, sulfoxides and various alkyl glycosides. The amount of absorption enhancer that can be included in the compositions of the present invention can be from about 0.01 to about 5 w %; preferably from about 0.5 to about 4 w % and most preferably from about 1 to about 2 w %. Examples of suitable antioxidants include without limitation ascorbyl palmitate, alpha tocopherol, butylated hydroxyanisole and fumaric acid. The amount of antioxidant that can be included in the compositions of the present invention can be from about 0.01 to about 20 w %; preferably from about 0.5 to about 10 w % or from about 0.5 to about 4 w % and most preferably from about 1 to about 2 w %.

The active substances include the active compounds selected from the group consisting of cyclosporine, sermorelin, octreotide acetate, calcitonin-salmon, insulin lispro, propofol succinate, clozepine, cyclobenzaprine, dexfenfluramine hydrochloride, glyburide, zidovudine, erythromycin, ciprofloxacin, ondansetron hydrochloride, dimenhydrinate, cimetidine hydrochloride, famotidine, phenyloin sodium, phenyloin, carboprost thromethamine, carboprost, diphenhydramine hydrochloride, isoproterenol hydrochloride, terbutaline sulfate, terbutaline, theophylline, albuterol sulfate and neutraceuticals, that is to say nutrients with pharmacological action such as but not limited to carnitine, valerian, echinacea, and the like.

In another embodiment, the active compound is a p-FOX (fatty acid oxidation) inhibitor, acetylcholinesterase inhibitor, nerve impulse inhibitor, anti-cholinergic, anti-convulsant, anti-psychotic, anxiolytic agent, dopamine metabolism inhibitor, agent to treat post stroke sequelae, neuroprotectant, agent to treat Alzheimer's disease, neurotransmitter, neurotransmitter agonist, sedative, agent for treating attention deficit disorder, agent for treating narcolepsy, central adregenic antagonist, anti-depression agent, agent for treating Parkinson's disease, benzodiazepine antagonist, stimulant, neurotransmitter antagonist, tranquilizer, or a mixture thereof.

In one embodiment the active compound is a p-FOX inhibitor. A suitable p-FOX inhibitor for use in the buccal sprays of the invention includes, but is not limited to, ranolazine.

In one embodiment the active compound is an acetylcholinesterase inhibitor. Suitable acetylcholinesterase inhibitors for use in the buccal sprays of the invention include, but are not limited to, galantamine, neostigmine, physostigmine, and edrophonium.

In one embodiment the active compound is a nerve impulse inhibitor. Suitable nerve impulse inhibitors for use in the buccal sprays of the invention include, but are not limited to, levobupivacaine, lidocaine, prilocaine, mepivacaine, propofol, rapacuronium bromide, ropivacaine, tubocurarine, atracurium, doxaurium, mivacurium, pancuronium, vercuronium, pipecuronium, and rocuronium.

In one embodiment the active compound is an anti-cholinergic. Suitable anti-cholinergics for use in the buccal sprays of the invention include, but are not limited to, amantadine, ipratropium, oxitropium, and dicycloverine.

In one embodiment the active compound is an anti-convulsant. Suitable anti-convulsants for use in the buccal sprays of the invention include, but are not limited to, acetazolamide, carbamazepine, clonazepam, diazepam, divalproex (valproic acid), ethosuximide, lamotrignine acid, levetriacetam, oxcarbazepine, phenobarbital, phenyloin, pregabalin, primidone, remacemide, trimethadione, topiramate, vigabatrin, and zonisamide.

In one embodiment the active compound is an anti-psychotic. Suitable anti-psychotics for use in the buccal sprays of the invention include, but are not limited to, amisulpride, aripiprazole bifemelane, bromperidol, clozapine, chlorpromazine, haloperidol, iloperidone loperidone, olanzapine, quetiapine, fluphenazine, fumarate, risperidone, thiothixene, thioridazine, sulpride, and ziprasidone,

In one embodiment the active compound is an anxiolytic agent. Suitable anxiolytic agents for use in the buccal sprays of the invention include, but are not limited to, amitryptyline, atracurium, buspirone, chlorzoxazone, clorazepate, cisatracurium, cyclobenzaprine, eperisone, esopiclone, hydroxyzine, mirtazapine, mivacurium, pagoclone, sulperide, zaleplon, and zopiclone.

In one embodiment the active compound is a dopamine metabolism inhibitor. Suitable dopamine metabolism inhibitors for use in the buccal sprays of the invention include, but are not limited to, entacapone, lazebemide, selegiline, and tolcapone.

In one embodiment the active compound is an agent to treat post stroke sequelae. Suitable agents to treat post stroke sequelae for use in the buccal sprays of the invention include, but are not limited to, glatiramer, interferon beta 1A, interferon beta 1B, estradiol, and progesterone.

In one embodiment the active compound is a neuroprotectant. Suitable neuroprotectants for use in the buccal sprays of the invention include, but are not limited to, donepezil, memanine, nimodipine, riluzole, rivastigmine, tacrine, TAK147, and xaliproden.

In one embodiment the active compound is an agent to treat Alzheimer's disease. Suitable agents to treat Alzheimer's disease for use in the buccal sprays of the invention include, but are not limited to, carbidopa, levodopa, tacrine, donezepil, rivastigmine, and galantamine.

In one embodiment the active compound is a neurotransmitter. Suitable neurotransmitters for use in the buccal sprays of the invention include, but are not limited to, acetylcholine, serotonin, 5-hydroxytryptamine (5-HT), GABA, glutamate, aspartate, glycine, histamine, epinephrine, norpinephrine, dopamine, adenosine, ATP, and nitric oxide.

In one embodiment the active compound is a neurotransmitter agonist. Suitable neurotransmitter agonists for use in the buccal sprays of the invention include, but are not limited to, almotriptan, aniracetam, atomoxetine, benserazide, bromocriptine, bupropion, cabergoline, citalopram, clomipramine, desipramine, diazepam, dihydroergotamine, doxepin duloxetine, eletriptan, escitalopram, fluvoxamine, gabapentin, imipramine, moclobemide, naratriptan, nefazodone, nefiracetam acamprosate, nicergoline, nortryptiline, paroxetine, pergolide, pramipexole, rizatriptan, ropinirole, sertraline, sibutramine, propofol, tiagabine, trazodone, venlafaxine, and zolmitriptan.

In one embodiment the active compound is a sedative. Suitable sedatives for use in the buccal sprays of the invention include, but are not limited to, dexmedetomidine, eszopiclone, indiplon, zolpidem, and zaleplon.

In one embodiment the active compound is an agent for treating attention deficit disorder. Suitable agents for treating attention deficit disorder for use in the buccal sprays of the invention include, but are not limited to, amphetamine, dextroamphetamine, methylphenidate, and pemoline.

In one embodiment the active compound is an agent for treating narcolepsy. Suitable agents for treating narcolepsy for use in the buccal sprays of the invention include, but are not limited to, modafinil and mazindol.

In one embodiment the active compound is a central adregenic antagonists. A suitable central adregenic antagonists for use in the buccal sprays of the invention includes, but is not limited to, mesoridazine.

In one embodiment the active compound is an anti-depression agent. Suitable anti-depression agents for use in the buccal sprays of the invention include, but are not limited to, amitriptyline, amoxapine, bupropion, clomipramine, clomipramine, clorgyline, desipramine, doxepin, fluoxetine, imipramine, isocarboxazid, maprotiline, mirtazapine, nefazodone, nortriptyline, paroxetine, phenelzine, protriptyline, sertraline, tranylcypromine, trazodone, and venlafaxine.

In one embodiment the active compound is an agent for treating Parkinson's disease. Suitable agents for treating Parkinson's disease for use in the buccal sprays of the invention include, but are not limited to, amantadine, bromocriptine, carvidopa, levodopa, pergolide, and selegiline.

In one embodiment the active compound is a benzodiazepine antagonist. A suitable benzodiazepine antagonist for use in the buccal sprays of the invention includes, but is not limited to, flumazenil.

In one embodiment the active compound is a neurotransmitter antagonist. A suitable neurotransmitter antagonist for use in the buccal sprays of the invention includes, but is not limited, to deramciclane.

In one embodiment the active compound is a stimulant. Suitable stimulants for use in the buccal sprays of the invention include, but are not limited to, amphetamine, dextroamphetamine, dinoprostone, methylphenidate, methylphenidate, modafinil, and pemoline.

In one embodiment the active compound is a tranquilizer. A suitable tranquilizer for use in the buccal sprays of the invention includes, but is not limited to, mesoridazine.

In one embodiment, the active compound of the compositions comprises propofol. Typically, when the active compound comprises propofol, the buccal spray composition contains propofol in an amount from about 0.1 to about 99.8 weight percent of the composition (w %), preferably about 1 to about 95 w % propofol, and more preferably about 5 to about 90 w % propofol. Also, the amounts of propofol can range from about 1 to about 80 w %, from about 1 to about 85 w %, from about 5 to about 75%, from about 10 to about 60 w % or from about 10 to about 70 w %.

Furthermore, when the buccal spray composition comprises propofol as an active compound, the solvent used in the composition can be a polar solvent, a non-polar solvent or a mixture thereof. Also, the buccal spray composition can be propellant free or it can contain a propellant. Moreover, the buccal spray composition that contains propofol as an active compound may contain a flavoring and/or masking agent.

The invention further relates to a method of administering propofol to a mammal in which the oral mucosa of the mammal is sprayed with a buccal spray composition comprising propofol.

The formulations of the present invention comprise an active compound or a pharmaceutically acceptable salt thereof. The term “pharmaceutically acceptable salts” refers to salts prepared from pharmaceutically acceptable non-toxic acids or bases including organic and inorganic acids or bases.

When an active compound of the present invention is acidic, salts may be prepared from pharmaceutically acceptable non-toxic bases. Salts derived from all stable forms of inorganic bases include aluminum, ammonium, calcium, copper, iron, lithium, magnesium, manganese, potassium, sodium, zinc, etc. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines and basic ion-exchange resins such as arginine, betaine, caffeine, choline, N,N dibenzylethylenediamine, diethylamine, 2-diethylaminoethanol, 2-dimethyl-aminoethanol, ethanolamine, ethylenediamine, N-ethylmorpholine, N-ethylpiperidine, glucamine, glucosamine, histidine, isopropylamine, lysine, methyl-glucosamine, morpholine, piperazine, piperidine, polyamine resins, procaine, purine, theobromine, triethylamine, trimethylamine, tripropylamine, etc.

When an active compound of the present invention is basic, salts may be prepared from pharmaceutically acceptable non-toxic acids. Such acids include acetic, benzenesulfonic, benzoic, camphorsulfonic, citric, ethane-sulfonic, fumaric, gluconic, glutamic, hydrobromic, hydrochloric, isethionic, lactic, maleic, mandelic, methanesulfonic, mucic, nitric, pamoic, pantothenic, phosphoric, succinic, sulfuric, tartaric, p-toluenesulfonic, etc. Particularly preferred are citric, hydrobromic, maleic, phosphoric, sulfuric, and tartaric acids.

In the discussion of methods of treatment herein, reference to the active compounds is meant to also include the pharmaceutically acceptable salts thereof. While certain formulations are set forth herein, the actual amounts to be administered to the mammal or man in need of same are to be determined by the treating physician.

The invention is further defined by reference to the following examples, which are intended to be illustrative and not limiting.

The following are examples of certain classes. All values unless otherwise specified are in weight percent.

### EXAMPLES

### Example 1

### Biologically Active Peptides Including Peptide Hormones

A. Cyclosporine Lingual Spray

| | | | most |
|---|---|---|---|
| | | preferred | preferred |
| | Amounts | amount | amount |
| cyclosporine | 5-50 | 10-35 | 15-25 |
| water | 5-20 | 7.5-50 | 9.5-12 |
| ethanol | 5-60 | 7.5-50 | 10-20 |
| polyethylene glycol | 20-60 | 30-45 | 35-40 |
| flavors | 0.1-5 | 1-4 | 2-3 |

B. Cyclosporine Non-Polar Lingual Spray

| | | | most |
|---|---|---|---|
| | | preferred | preferred |
| | Amounts | amount | amount |
| cyclosporine | 1-50 | 3-40 | 5-30 |
| Migylol | 20 | 25 | 30-40 |
| Polyoxyethylated | 20 | 25 | 30-40 |
| castor oil | | | |
| Butane | 25-80 | 30-70 | 33-50 |
| flavors | 0.1-5 | 1-4 | 2-3 |

C. Cyclosporine Non-Polar Bite Capsule

| | | | most |
|---|---|---|---|
| | | preferred | preferred |
| | Amounts | amount | amount |
| cyclosporine | 1-35 | 5-25 | 10-20 |
| olive oil | 25-60 | 35-55 | 30-45 |
| polyoxyethylated | 25-60 | 35-55 | 30-45 |
| oleic glycerides | | | |
| flavors | 0.1-5 | 1-4 | 2-3 |

D. Cyclosporine Bite Capsule

| | | | most |
|---|---|---|---|
| | | preferred | preferred |
| | Amounts | amount | amount |
| cyclosporine | 5-50 | 10-35 | 15-25 |
| polyethylene | 20-60 | 30-45 | 35-40 |
| glycol | | | |
| glycerin | 5-30 | 7.5-25 | 10-20 |
| propylene glycol | 5-30 | 7.5-25 | 10-20 |
| flavors | 0.1-10 | 1-8 | 3-6 |

E. Sermorelin (as the Acetate) Lingual Spray

| | | preferred | most |
|---|---|---|---|
| | Amounts | amount | preferred |
| sermorelin (as the acetate) | .01-5 | .1-3 | .2-1.0 |
| mannitol | 1-25 | 5-20 | 10-15 |
| monobasic sodium phosphate, | 0.1-5 | 1-31 | .5-2.5 |
| dibasic sodium phosphate water | 0.01-5 | .05-3 | 0.1-0.5 |
| ethanol | 5-30 | 7.5-25 | 9.5-15 |
| polyethylene glycol | 20-60 | 30-45 | 35-40 |
| propylene glycol | 5-25 | 10-20 | 12-17 |
| flavors | 0.1-5 | 1-4 | 2-3 |

F. Octreotide Acetate (Sandostatin) Lingual Spray

| | | | most |
|---|---|---|---|
| | | preferred | preferred |
| | Amounts | amount | amount |
| octreotide acetate | 0.001-0.5 | 0.005-0.250 | 0.01-0.10 |
| acetic acid | 1-10 | 2-8 | 4-6 |
| sodium acetate | 1-10 | 2-8 | 4-6 |
| sodium chloride | 3-30 | .5-25 | 15-20 |
| flavors | 0.1-5 | 0.5-.4 | 2-3 |
| ethanol | 5-30 | 7.5-20 | 9.5-15 |
| water | 15-95 | 35-90 | 65-85 |
| flavors | 0.1-5 | 1-4 | 2-3 |

G. Calcitonin-Salmon Lingual Spray

| | | | most |
|---|---|---|---|
| | | preferred | preferred |
| | Amounts | amount | amount |
| calcitonin-salmon | 0.001-5 | 0.005-2 | 01-1.5 |
| ethanol | 2-15 | 3-10 | 7-9.5 |
| water | 30-95 | 50-90 | 60-80 |
| polyethylene | 2-15 | 3-10 | 7-9.5 |
| glycol | | | |
| sodium chloride | 2.5-20 | 5-15 | 10-12.5 |
| flavors | 0.1-5 | 1-4 | 2-3 |

H. Insulin Lispro, Lingual Spray

| | | | most |
|---|---|---|---|
| | | preferred | preferred |
| | Amounts | amount | amount |
| insulin | 20-60 | 4-55 | 5-50 |
| glycerin | 0.1-10 | 0.25-5 | 0.1-1.5 |
| dibasic sodium | 1-15 | 2.5-10 | 4-8 |
| phosphate | | | |
| m-cresol, | 1-25 | 5-25 | 7.5-12.5 |
| zinc oxide | 0.01-0.25 | .05-0.15 | 0.075-0.10 |
| m-cresol | 0.1-1 | 0.2-0.8 | 0.4-0.6 |
| phenol | trace amounts | trace amounts | trace amounts |
| ethanol | 5-20 | 7.5-15 | 9-12 |
| water | 30-90 | 40-80 | 50-75 |
| propylene glycol | 5-20 | 7.5-15 | 9-12 |
| flavors | 0.1-5 | 0.5-3 | 0.75-2 |

adjust pH to 7.0-7.8 with HCI or NaOH

### Example 2

CNS active amines and their salts: including but not limited to tricyclic amines, GABA analogues, thiazides, phenothiazine derivatives, serotonin antagonists and serotonin reuptake inhibitors

A. Sumatriptan Succinate Lingual Spray

| | | | most |
|---|---|---|---|
| | | preferred | preferred |
| | Amounts | amount | amount |
| Sumatriptan succinate | 0.5-30 | 1-20 | 10-15 |
| Ethanol | 5-60 | 7.5-50 | 10-20 |
| propylene glycol | 5-30 | 7.5-20 | 10-15 |
| polyethylene glycol | 0-60 | 30-45 | 35-40 |
| Water | 5-30 | 7.5-20 | 10-15 |
| Flavors | 0.1-5 | 1-4 | 2-3 |

B. Sumatriptan Sucinate Bite Capsule

| | | | most |
|---|---|---|---|
| | | preferred | preferred |
| | Amounts | amount | amount |
| Sumatriptan | 0.01-5 | 0.05-3.5 | 0.075-1.75 |
| succinate | | | |
| polyethylene | 25-70 | 30-60 | 35-50 |
| glycol | | | |
| Glycerin | 25-70 | 30-60 | 35-50 |
| Flavors | 0.1-10 | 1-8 | 3-6 |

C. Clozepine Lingual Spray

| | | | most |
|---|---|---|---|
| | | preferred | preferred |
| | Amounts | amount | amount |
| Clozepine | 0.5-30 | 1-20 | 10-15 |
| Ethanol | 5-60 | 7.5-50 | 10-20 |
| propylene glycol | 5-30 | 7.5-20 | 10-15 |
| polyethylene glycol | 0-60 | 30-45 | 35-40 |
| Water | 5-30 | 7.5-20 | 10-15 |
| Flavors | 0.1-5 | 1-4 | 2-3 |

D. Clozepine Non-Polar Lingual Spray with Propellant

| | | | most |
|---|---|---|---|
| | | preferred | preferred |
| | Amounts | amount | amount |
| Clozepine | 0.5-30 | 1-20 | 10-15 |
| Migylol | 20-85 | 25-70 | 30-40 |
| Butanol | 5-80 | 30-75 | 60-70 |
| Flavors | 0.1-5 | 1-4 | 2-3 |

E. Clozepine Non-Polar Lingual Spray without Propellant

| | | | most |
|---|---|---|---|
| | | preferred | preferred |
| | Amounts | amount | amount |
| Clozepine | 0.5-30 | 1-20 | 10-15 |
| Migylol | 70-99.5 | 80-99 | 85-90 |
| Flavors | 0.1-5 | 1-4 | 2-3 |

F Cyclobenzaprine Non-Polar Lingual Spray

| | | | most |
|---|---|---|---|
| | | preferred | preferred |
| | Amounts | amount | amount |
| cyclobenzaprine (base) | 0.5-30 | 1-20 | 10-15 |
| Migylol | 20-85 | 25-70 | 30-40 |
| Iso-butane | 15-80 | 30-75 | 60-70 |
| Flavors | 0.1-5 | 1-4 | 2-3 |

G. Dexfenfluramine Hydrochloride Lingual Spray

| | | | most |
|---|---|---|---|
| | | preferred | preferred |
| | Amounts | amount | amount |
| dexfenfluramine Hcl | 5-30 | 7.5-20 | 10-15 |
| Ethanol | 5-60 | 7.5-50 | 10-20 |
| propylene glycol | 5-30 | 7.5-20 | 10-15 |
| Polyethylene glycol | 0-60 | 30-45 | 35-40 |
| Water | 5-30 | 7.5-20 | 10-15 |
| Flavors | 0.1-5 | 1-4 | 2-3 |

### Example 3

### Sulfonylureas

A. Glyburide Lingual Spray

| | | | most |
|---|---|---|---|
| | | preferred | preferred |
| | Amounts | amount | amount |
| Glyburide | 0.25-25 | 0.5-20 | 0.75-15 |
| Ethanol | 5-60 | −7.5-50 | 10-20 |
| propylene glycol | 5-30 | 7.5-20 | 10-15 |
| Polyethylene glycol | 0-60 | 30-45 | 35-40 |
| Water | 2.5-30 | 5-20 | 6-15 |
| Flavors | 0.1-5 | 1-4 | 2-3 |

B. Glyburide Non-Polar Bite Capsule

| | | | most |
|---|---|---|---|
| | | preferred | preferred |
| | Amounts | amount | amount |
| Glyburide | 0.01-10 | 0.025-7.5 | 0.1-4 |
| olive oil | 30-60 | 35-55 | 30-50 |
| polyoxyethylated | 30-60 | 35-55 | 30-50 |
| oleic glycerides | | | |
| Flavors | 0.1-5 | 1-4 | 2-3 |

### Example 4

### Antibiotics Anti-Fungals and Anti-Virals

A. Zidovudine [Formerly Called Azidothymidine (AZT) (Retrovir)] Non-Polar Lingual Spray

| | | | most |
|---|---|---|---|
| | | preferred | preferred |
| | Amounts | amount | amount |
| Zidovudine | 10-50 | 15-40 | 25-35 |
| Soya oil | 20-85 | 25-70 | 30-40 |
| Butane | 15-80 | 30-75 | 60-70 |
| Flavors | 0.1-5 | 1-4 | 2-3 |

B. Erythromycin Bite Capsule Bite Capsule

| | | | most |
|---|---|---|---|
| | | preferred | preferred |
| | Amounts | amount | amount |
| Erythromycin | 25-65 | 30-50 | 35-45 |
| polyoxyethylene | 5-70 | 30-60 | 45-55 |
| glycol | | | |
| Glycerin | 5-20 | 7.5-15 | 10-12.5 |
| Flavors | 1-10 | 2-8 | 3-6 |

C. Ciprofloxacin Hydrochloride Bite Capsule

| | | | most |
|---|---|---|---|
| | | preferred | preferred |
| | Amounts | amount | amount |
| Ciprofloxacin | 25-65 | 35-55 | 40-50 |
| hydrochloride | | | |
| Glycerin | 5-20 | 7.5-15 | 10-12.5 |
| Polyethylene glycol | 120-75 | 30-65 | 40-60 |
| Flavors | 1-10 | 2-8 | 3-6 |

D. Zidovudine [Formerly Called Azidothymidine (AZT) (Retrovir)] Lingual Spray

| | | | most |
|---|---|---|---|
| | | preferred | preferred |
| | Amounts | amount | amount |
| Zidovudine | 10-50 | 15-40 | 25-35 |
| Water | 30-80 | 40-75 | 45-70 |
| Ethanol | 5-20 | 7.5-15 | 9.5-12.5 |
| polyethylene glycol | 5-20 | 7.5-15 | 9.5-12.5 |
| Flavors | 0.1-5 | 1-4 | 2-3 |

### Example 5

### Anti-Emetics

A. Ondansetron Hydrochloride Lingual Spray

| | | | most |
|---|---|---|---|
| | | preferred | preferred |
| | Amounts | amount | amount |
| ondansetron hydrochloride | 1-25 | 2-20 | 2.5-15 |
| citric acid monohydrate | 1-10 | 2-8 | 2.5-5 |
| sodium citrate dihydrate | 0.5-5 | 1-4 | 1.25-2.5 |
| Water | 1-90 | 5-85 | 10-75 |
| Ethanol | 5-30 | 7.5-20 | 9.5-15 |
| propylene glycol | 5-30 | 7.5-20 | 9.5-15 |
| polyethylene glycol | 5-30 | 7.5-20 | 9.5-15 |
| Flavors | 1-10 | 3-8 | 5-7.5 |

B. Dimenhydrinate Bite Capsule

| | | | most |
|---|---|---|---|
| | | preferred | preferred |
| | Amounts | amount | amount |
| dimenhydrinate | 0.5-30 | 2-25 | 3-15 |
| Glycerin | 5-20 | 7.5-15 | 10-12.5 |
| polyethylene glycol | 45-95 | 50-90 | 55-85 |
| Flavors | 1-10 | 2-8 | 3-6 |

C. Dimenhydrinate Polar Lingual Spray

| | | | most |
|---|---|---|---|
| | | preferred | preferred |
| | Amounts | amount | amount |
| dimenhydrinate | 3-50 | 4-40 | 5-35 |
| Water | 5-90 | 10-80 | 15-75 |
| Ethanol | 1-80 | 3-50 | 5-10 |
| polyethylene glycol | 1-80 | 3-50 | 5-15 |
| Sorbitol | 0.1-5 | 0.2-40 | 0.4-1.0 |
| Aspartame | 0.01-0.5 | 0.02-0.4 | 0.04-0.1 |
| Flavors | 0.1-5 | 1-4 | 2-3 |

### Example 6

### Histamine H-2 Receptor Antagonists

A. Cimetidine Hydrochloride Bite Capsule

| | | | most |
|---|---|---|---|
| | | preferred | preferred |
| | Amounts | amount | amount |
| cimetidine HCl | 10-60 | 15-55 | 25-50 |
| Glycerin | 5-20 | 7.5-15 | 10-12.5 |
| Polyethylene glycol | 20-90 | 25-85 | 30-75 |
| Flavors | 1-10 | 2-8 | 3-6 |

B. Famotidine Lingual Spray

| | | | most |
|---|---|---|---|
| | | preferred | preferred |
| | Amounts | amount | amount |
| Famotidine | 1-35 | 5-30 | 7-20 |
| Water | 2.5-25 | 3-20 | 5-10 |
| L-aspartic acid | 0.1-20 | 1-15 | 5-10 |
| Polyethylene glycol | 20-97 | 30-95 | 50-85 |
| Flavors | 0.1-10 | 1-7.5 | 2-5 |

C. Famotidine Non-Polar Lingual Spray

| | | | most |
|---|---|---|---|
| | | preferred | preferred |
| | Amounts | amount | amount |
| Famotidine | 1-35 | 5-30 | 7-20 |
| Soya oil | 10-50 | 15-40 | 15-20 |
| Butane1 | 5-80 | 30-75 | 45-70 |
| polyoxyethylated | 10-50 | 15-40 | 15-20 |
| oleic glycerides | | | |
| Flavors | 0.1-5 | 1-4 | 2-3 |

### Example 7

### Barbiturates

A. Phenyloin Sodium Lingual Spray

| | | | most |
|---|---|---|---|
| | | preferred | preferred |
| | Amounts | amount | amount |
| phenytoin sodium | 10-60 | 15-55 | 20-40 |
| Water | 2.5-25 | 3-20 | 5-10 |
| Ethanol | 5-30 | 7.5-20 | 9.5-15 |
| propylene glycol | 5-30 | 7.5-20 | 9.5-15 |
| Polyethylene glycol | 5-30 | 7.5-20 | 9.5-15 |
| Flavors | 1-10 | 3-8 | 5-7.5 |

D. Phenyloin Non-Polar Lingual Spray

| | | | most |
|---|---|---|---|
| | | preferred | preferred |
| | Amounts | amount | amount |
| Phenytoin | 5-45 | 10-40 | 15-35 |
| Migylol | 10-50 | 15-40 | 15-20 |
| Butane | 15-80 | 30-75 | 60-70 |
| polyoxyethylated | 10-50 | 15-40 | 15-20 |
| oleic glycerides | | | |
| Flavors | 0.1-10 | 1-8 | 5-7.5 |

### Example 8

### Prostaglandins

A. Carboprost Thromethamine Lingual Spray

| | | | most |
|---|---|---|---|
| | | preferred | preferred |
| | Amounts | amount | amount |
| carboprost | 0.05-5 | 0.1-3 | 0.25-2.5 |
| thromethamine | | | |
| Water | 50-95 | 60-80 | 65-75 |
| Ethanol | 5-20 | 7.5-15 | 9.5-12.5 |
| Polyethylene glycol | 5-20 | 7.5-15 | 9.5-12.5 |
| sodium chloride | 1-20 | 3-15 | 4-8 |
| Flavors | 0.1-5 | 1-4 | 2-3 |

pH is adjusted with sodium hydroxide and/or hydrochloric acid

B. Carboprost Non-Polar Lingual Spray

| | | | most |
|---|---|---|---|
| | | preferred | preferred |
| | Amounts | amount | amount |
| Carboprost | 0.05-5 | 0.1-3 | 0.25-2.5 |
| Migylol | 25-50 | 30-45 | 35-40 |
| Butane | 5-60 | 10-50 | 20-35 |
| polyoxyethylated | 25-50 | 30-45 | 35-40 |
| oleic glycerides | | | |
| Flavors | 0.1-10 | 1-8 | 5-7.5 |

### Example 9

### Neutraceuticals

A. Carnitine as Bite Capsule (Contents are a Taste)

| | | | most |
|---|---|---|---|
| | | preferred | preferred |
| | Amounts | amount | amount |
| carnitine fumarate | 6-80 | 30-70 | 45-65 |
| soya oil | 7.5-50 | 10-40 | 12.5-35 |
| soya lecithin | 0.001-1.0 | 0.005-0.5 | .01-0.1 |
| Soya fats | 7.5-50 | 10-40 | 12.5-35 |
| Flavors | 1-10 | 2-8 | 3-6 |

B. Valerian as Lingual Spray

| | | | most |
|---|---|---|---|
| | | preferred | preferred |
| | Amounts | amount | amount |
| valerian extract | 0.1-10 | 0.2-7 | 0.25-5 |
| Water | 50-95 | 60-80 | 65-75 |
| Ethanol | 5-20 | 7.5-15 | 9.5-12.5 |
| Polyethylene glycol | 5-20 | 7.5-15 | 9.5-12.5 |
| Flavors | 1-10 | 2-8 | 3-6 |

C. Echinacea as Bite Capsule

| | | | most |
|---|---|---|---|
| | | preferred | preferred |
| | Amounts | amount | amount |
| echinacea extract | 30-85 | 40-75 | 45-55 |
| soya oil | 7.5-50 | 10-40 | 12.5-35 |
| soya lecithin | 0.001-1.0 | 0.005-0.5 | .01-0.1 |
| Soya fats | 7.5-50 | 10-40 | 12.5-35 |
| Flavors | 1-10 | 2-8 | 3-6 |

D. Mixtures of Ingredients

| | | | most |
|---|---|---|---|
| | | preferred | preferred |
| | Amounts | amount | amount |
| magnesium oxide | 15-40 | 20-35 | 25-30 |
| chromium picolinate | 0.01-1.0 | 0.02-0.5 | .025-0.75 |
| folic acid | .025-3.0 | 0.05-2.0 | 0.25-0.5 |
| vitamin B-12 | 0.01-1.0 | 0.02-0.5 | .025-0.75 |
| vitamin E | 15-40 | 20-35 | 25-30 |
| Soya oil | 10-40 | 12.5-35 | 15-20 |
| soya lecithin | 0.1-5 | 0.2-4 | 0.5-1.5 |
| soya fat | 10-40 | 15-35 | 17.5-20 |

### Example 10

### Sleep Inducers (Also CNS Active Amine)

A. Diphenhydramine Hydrochloride Lingual Spray

| | | | most |
|---|---|---|---|
| | | preferred | preferred |
| | Amounts | amount | amount |
| diphenhydramine | 3-50 | 4-40 | 5-35 |
| HCl water | 5-90 | 10-80 | 50-75 |
| Ethanol | 1-80 | 3-50 | 5-10 |
| Polyethylene glycol | 1-80 | 3-50 | 5-15 |
| Sorbitol | 0.1-5 | 0.2-4 | 0.4-1.0 |
| Aspartame | 0.01-0.5 | 0.02-0.4 | 0.04-0.1 |
| Flavors | 0.1-5 | 1-4 | 2-3 |

### Example 11

### Anti-Asthmatics-Bronchodilators

A. Isoproterenol Hydrochloride as Polar Lingual Spray

| | | | most |
|---|---|---|---|
| | | preferred | preferred |
| | Amounts | amount | amount |
| Isoproterenol | 0.1-10 | 0.2-7.5 | 0.5-6 |
| Hydrochloride | | | |
| Water | 5-90 | 10-80 | 50-75 |
| Ethanol | 1-80 | 3-50 | 5-10 |
| Polyethylene glycol | 1-80 | 3-50 | 5-15 |
| Sorbitol | 0.1-5 | 0.2-4 | 0.4-1.0 |
| Aspartame | 0.01-0.5 | 0.02-0.4 | 0.04-0.1 |
| Flavors | 0.1-5 | 1-4 | 2-3 |

B. Terbutaline Sulfate as Polar Lingual Spray

| | | | most |
|---|---|---|---|
| | | preferred | preferred |
| | Amounts | amount | amount |
| terbutaline sulfate | 0.1-10 | 0.2-7.5 | 0.5-6 |
| Water | 5-90 | 10-80 | 50-75 |
| Ethanol | 1-10 | 2-8 | 2.5-5 |
| Sorbitol | 0.1-5 | 0.2-4 | 0.4-1.0 |
| Aspartame | 0.01-0.5 | 0.02-0.4 | 0.04-0.1 |
| Flavors | 0.1-5 | 1-4 | 2-3 |

C. Terbutaline as Non-Polar Lingual Spray

| | | | most |
|---|---|---|---|
| | | preferred | preferred |
| | Amounts | amount | amount |
| Terbutaline | 0.1-10 | 0.2-7.5 | 0.5-6 |
| Migylol | 25-50 | 30-45 | 35-40 |
| Isobutane | 5-60 | 10-50 | 20-35 |
| polyoxyethylated | 25-50 | 30-45 | 35-40 |
| oleic glycerides | | | |
| Flavors | 0.1-10 | 1-8 | 5-7.5 |

D. Theophylline Polar Bite Capsule

| | | | most |
|---|---|---|---|
| | | preferred | preferred |
| | Amounts | amount | amount |
| Theophylline | 5-50 | 10-40 | 15-30 |
| Polyethylene glycol | 20-60 | 25-50 | 30-40 |
| Glycerin | 25-50 | 35-45 | 30-40 |
| propylene glycol | 25-50 | 35-45 | 30-40 |
| Flavors | 0.1-5 | 1-4 | 2-3 |

E. Albuterol Sulfate as Polar Lingual Spray

| | Amounts | preferred amount | most preferred amount |
|---|---|---|---|
| albuterol sulfate | 0.1-10 | 0.2-7.5 | 0.5-6 |
| Water | 5-90 | 10-80 | 50-75 |
| Ethanol | 1-10 | 2-8 | 2.5-5 |
| Sorbitol | 0.1-5 | 0.2-4 | 0.4-1.0 |
| Aspartame | 0.01-0.5 | 0.02-0.4 | 0.04-0.1 |
| Flavors | 0.1-5 | 1-4 | 2-3 |

### Example 12

### Polar Solvent Formulations Using a Propellant

A. Sulfonylurea

| | | Preferred | Most-Preferred |
|---|---|---|---|
| | Amount | Amount | Amount |
| Glyburide | 0.1-25% | 0.5-15% | 0.6-10% |
| Ethanol | 40-99% | 60-97% | 70-97% |
| Water | 0.01-5% | 0.1-4% | 0.2-2% |
| Flavors | 0.05-10% | 0.1-5% | 0.1-2.5% |
| Propellant | 2-10% | 3-5% | 3-4% |

B. Prostaglandin E (Vasodilator)

| | | Preferred | Most-Preferred |
|---|---|---|---|
| | Amount | Amount | Amount |
| prostaglandin | 0.01-10% | 0.1-5% | 0.2-3% |
| E₁ | | | |
| Ethanol | 10-90% | 20-75% | 25-50% |
| Propylene | 1-90% | 5-80% | 10-75% |
| glycol | | | |
| Water | 0.01-5% | 0.1-4% | 0.2-2% |
| Flavors | 0.05-10% | 0.1-5% | 0.1-2.5% |
| Propellant | 2-10% | 3-5% | 3-4% |

C. Promethazine (Antiemetic, Sleep Inducer, and CNS Active Amine)

| | | Preferred | Most-Preferred |
|---|---|---|---|
| | Amount | Amount | Amount |
| Promethazine | 1-25% | 3-15% | 5-12% |
| Ethanol | 10-90% | 20-75% | 25-50% |
| Propylene | 1-90% | 5-80% | 10-75% |
| glycol | | | |
| Water | 0.01-5% | 0.1-4% | 0.2-2% |
| Flavors | 0.05-10% | 0.1-5% | 0.1-2.5% |
| Propellant | 2-10% | 3-5% | 3-4% |

D. Meclizine

| | | Preferred | |
|---|---|---|---|
| | Amount | Amount | Most-Preferred Amount |
| Meclizine | 1-25% | 3-15% | 5-12% |
| Ethanol | 1-15% | 2-10% | 3-6 |
| Propylene glycol | 20-98% | 5-90% | 10-85% |
| Water | 0.01-5% | 0.1-4% | 0.2-2% |
| Flavors | 0.05-10% | 0.1-5% | 0.1-2.5% |
| Propellant | 2-10% | 3-5% | 3-4% |

### Example 13

### Buccal Spray Formulations Comprising Propofol

A. Exemplary Components for Propellant Free Buccal Spray Formulations Comprising Propofol in a Polar Solvent

| | | Preferred | Most Preferred |
|---|---|---|---|
| Components | Amount (w %) | Amount (w %) | Amount (w %) |
| Propofol | 0.1 to 99.8 | 1 to 95 | 5 to 90 |
| Oleic Acid | 0.01 to 5 | 0.5 to 4 | 1 to 2 |
| Flavoring | 0.01 to 5 | 0.5 to 4 | 1 to 2 |
| agent/taste | | | |
| mask | | | |
| Ascorbyl | 0.01 to 20 | 0.5 to 10 | 1 to 2 |
| Palmitate | | | |
| Ethanol USP | 0.05 to 98.7 | 1 to 75 | 5 to 60 |

1. A propellant free buccal spray propofol formulation comprising propofol in a polar solvent contained the following:

| | | Weight Percent of |
|---|---|---|
| Components | Amount (g) | Composition (w %) |
| Propofol | 80 | 85.7 |
| Oleic Acid | 1 | 1.1 |
| Bitter Mask | 1 | 1.1 |
| Ascorbyl Palmitate | 2 | 2.1 |
| Ethanol USP | 9.38 | 10.0 |
| Total | 93.38 | 100.0 |

2. A propellant free buccal spray propofol formulation comprising propofol in a polar solvent contained the following:

| | | Weight Percent of |
|---|---|---|
| Components | Amount (g) | Composition (w %) |
| Propofol | 90 | 95.3 |
| Oleic Acid | 1 | 1.1 |
| Bitter Mask | 1 | 1.1 |
| Ascorbyl Palmitate | 0.2 | 0.2 |
| Ethanol USP | 2.21 | 2.3 |
| Total | 94.41 | 100.0 |

B. Exemplary Components for Buccal Spray Formulations Comprising a Propellant and Propofol in a Polar Solvent

| | | Preferred | Most Preferred |
|---|---|---|---|
| Components | Amount (w %) | Amount (w %) | Amount (w %) |
| Propofol | 1 to 85 | 5 to 75 | 10 to 70 |
| Oleic Acid | 0.01 to 5 | 0.5 to 4 | 1 to 2 |
| Flavoring | 0.01 to 5 | 0.5 to 4 | 1 to 2 |
| agent/taste | | | |
| mask | | | |
| Ascorbyl | 0.01 to 20 | 0.5 to 10 | 1 to 2 |
| Palmitate | | | |
| Ethanol | 1 to 85 | 5 to 75 | 10 to 60 |
| Butane | 10 to 90 | 10 to 85 | 15 to 65 |

C. Exemplary Components for Propellant Free Buccal Spray Formulations Comprising Propofol in a Non-Polar Solvent

| | | Preferred | Most Preferred |
|---|---|---|---|
| Components | Amount (w %) | Amount (w %) | Amount (w %) |
| Propofol | 0.1 to 99.8 | 1 to 95 | 5 to 90 |
| Miglyol 810 | 0.05 to 98.7 | 1 to 75 | 5 to 60 |
| Oleic Acid | 0.01 to 5 | 0.5 to 4 | 1 to 2 |
| Flavoring | 0.01 to 5 | 0.5 to 4 | 1 to 2 |
| agent/taste | | | |
| mask | | | |
| Ascorbyl | 0.01 to 20 | 0.5 to 10 | 1 to 2 |
| Palmitate | | | |

D. Exemplary Components for Buccal Spray Formulations Comprising a Propellant and Propofol in a Non-Polar Solvent

| | | Preferred | Most Preferred Amount |
|---|---|---|---|
| Components | Amount (w %) | Amount (w %) | (w %) |
| Propofol | 1 to 85 | 5 to 75 | 10 to 70 |
| Oleic Acid | 0.01 to 5 | 0.5 to 4 | 1 to 2 |
| Flavoring | 0.01 to 5 | 0.5 to 4 | 1 to 2 |
| agent/taste | | | |
| mask | | | |
| Ascorbyl | 0.01 to 20 | 0.5 to 10 | 1 to 2 |
| Palmitate | | | |
| Miglyol 810 | 1 to 85 | 5 to 75 | 10 to 60 |
| Butane | 10 to 90 | 10 to 85 | 15 to 65 |

E. Exemplary Components for Propellant Free Buccal Spray Formulations Comprising Propofol in a Mixture of a Polar and a Non-Polar Solvents

| | | Preferred | Most Preferred |
|---|---|---|---|
| Components | Amount (w %) | Amount (w %) | Amount (w %) |
| Propofol | 0.1 to 99.8 | 1 to 95 | 5 to 90 |
| Miglyol 810 | 0.1 to 80 | 0.5 to 75 | 1 to 60 |
| Oleic Acid | 0.01 to 5 | 0.5 to 4 | 1 to 2 |
| Flavoring | 0.01 to 5 | 0.5 to 4 | 1 to 2 |
| agent/taste | | | |
| mask | | | |
| Ascorbyl | 0.01 to 20 | 0.5 to 10 | 1 to 2 |
| Palmitate | | | |
| Ethanol USP | 0.05 to 98.7 | 1 to 75 | 5 to 60 |

1. A propellant free buccal spray formulation comprising propofol in a mixture of polar and non-polar solvents contained the following:

| | | Weight Percent of |
|---|---|---|
| Components | Amount (g) | Composition (w %) |
| Propofol | 20 | 22.3 |
| Miglyol 810 | 40 | 44.6 |
| Oleic Acid | 1 | 1.1 |
| Bitter Mask | 1 | 1.1 |
| Ascorbyl Palmitate | 0.4 | 0.4 |
| Ethanol USP | 27.2 | 30.4 |
| Total | 89.6 | 100.0 |

Samples of this buccal spray formulation were tested to see if exposure of the samples to long-term stability conditions as well as accelerated stability conditions affected certain chemical and physical properties of the samples. The samples were tested using commercially-available single dose actuators. Long term stability conditions were defined as 25±2 degrees centigrade and 60±5% relative humidity. Accelerated stability conditions were defined as 40±2 degrees centigrade and 75±5% relative humidity. The samples were stored both in horizontal and upright orientations. The spray volumes, content, uniformity, pattern, angle and droplet size distribution of samples were determined at one month after imposing both long term and accelerated stability conditions on the samples. Each assay was performed 5 times, except for droplet size distribution which was performed 15 times. The spray parameters were defined to be within specification if the coefficient of variation from the base line was 5% or less. It was determined that the tested physical and chemical properties remained within specification after exposure of the samples to the long-term and accelerated stability conditions.

2. A propellant free buccal spray propofol formulation comprising propofol in a mixture of polar and non-polar solvents contained the following:

| | | Weight Percent of |
|---|---|---|
| Components | Amount (g) | Composition (w %) |
| Propofol | 60 | 64.5 |
| Miglyol 810 | 20 | 21.5 |
| Oleic Acid | 1 | 1.1 |
| Bitter Mask | 1 | 1.1 |
| Ascorbyl Palmitate | 0.2 | 0.2 |
| Ethanol USP | 10.78 | 11.6 |
| Total | 92.98 | 100.0 |

F. Exemplary Components for Buccal Spray Formulations Comprising a Propellant and Propofol in a Mixture of Polar and Non-Polar Solvents

| | | Preferred | Most Preferred Amount |
|---|---|---|---|
| Components | Amount (w %) | Amount (w %) | (w %) |
| Propofol | 1 to 80 | 5 to 75 | 10 to 60 |
| Oleic Acid | 0.01 to 5 | 0.5 to 4 | 1 to 2 |
| Flavoring | 0.01 to 5 | 0.5 to 4 | 1 to 2 |
| agent/taste | | | |
| mask | | | |
| Ascorbyl | 0.01 to 20 | 0.5 to 10 | 1 to 2 |
| Palmitate | | | |
| Miglyol 810 | 1 to 80 | 2 to 75 | 10 to 60 |
| Ethanol | 2 to 80 | 5 to 75 | 10 to 60 |
| Butane | 10 to 90 | 10 to 85 | 15 to 65 |

1-109. (canceled)

110. A method of administering propofol to a mammal, comprising spraying the oral mucosa of the mammal with a propellant free buccal spray composition to provide transmucosal absorption of a therapeutically effective amount of propofol through the oral mucosa of the mammal to the systemic circulatory system of the mammal, the composition comprising: propofol in an amount of between about 0.1 and about 99.8 percent by weight of the total composition; and a polar solvent in an amount between about 0.05 and about 98.7 percent by weight of the total composition, wherein a therapeutically effective amount of propofol is absorbed through the oral mucosa of the mammal to the mammal's systemic circulatory system.

111. The method of claim 110, further comprising a taste mask and/or flavoring agent in an amount of between about 0.01 and about 5 percent by weight of the total composition.

112. The method of claim 111, wherein the propofol is present in an amount between about 1 and about 95 percent by weight of the total composition, the polar solvent is present in an amount between about 1 and about 75 percent by weight of the total composition, and the taste mask and/or flavoring agent is present in an amount between about 0.5 and about 4 percent by weight of the total composition.

113. The method of claim 112, wherein the propofol is present in an amount between about 5 and about 90 percent by weight of the total composition, the polar solvent is present in an amount between about 5 and about 60 percent by weight of the total composition, and the taste mask and/or flavoring agent is present in an amount between about 1 and about 2 percent by weight of the total composition.

114. The method of claim 110, wherein the solvent comprises a polyethylene glycol having a molecular weight between 400 and 1000, C₂ to C₈ mono- and poly-alcohol, or C₇ to C₁₈ alcohol of linear or branched configuration.

115. The method of claim 110, wherein the solvent comprises polyethylene glycol.

116. The method of claim 110, wherein the solvent comprises ethanol.

117. The method of claim 111, wherein the flavoring agent comprises a synthetic or natural oil of peppermint, oil of spearmint, citrus oil, fruit flavor, sweetener, or mixture thereof.

118. The method of claim 110, further comprising an absorption enhancer in an amount of between about 0.01 to about 5 percent by weight of the total composition.

119. The method of claim 112, further comprising an absorption enhancer in an amount of between about 0.5 to about 4 percent by weight of the total composition.

120. The method of claim 113, further comprising an absorption enhancer in an amount of between about 1 to about 2 percent by weight of the total composition.

121. The method of claim 118, wherein the absorption enhancer comprises oleic acid, 23-lauryl ether, aprotinin, azone, benzalkonium chloride, cetylpyridinium chloride, cetyltrimethylammonium bromide, cyclodextrin, dextran sulfate, lauric acid, lauric acid/propylene glycol, lysophosphatidylcholine, menthol, methoxysalicylate, methyloleate, phosphatidylcholine, polyoxyethylene, polysorbate 80, sodium EDTA (ethylenediamine tetraacetic acid), sodium glycocholate, sodium glycodeoxycholate, sodium lauryl sulfate, sodium salicylate, sodium taurocholate, sodium taurodeoxycholate, sulfoxides or an alkyl glycoside.

122. The method of claim 110, further comprising an antioxidant in an amount of between about 0.01 to about 20 percent by weight of the total composition.

123. The method of claim 112, further comprising an antioxidant in an amount of between about 0.5 to about 10 percent by weight of the total composition.

124. The method of claim 113, further comprising an antioxidant in an amount of between about 1 to about 2 percent by weight of the total composition.

125. The method of claim 122, wherein the antioxidant comprises ascorbyl palmitate, alpha tocopherol, butylated hydroxyanisole or fumaric acid.

126. The method of claim 110, wherein the amount of the spray is predetermined.

127. A method of administering propofol to a mammal, comprising spraying the oral mucosa of the mammal with a propellant free buccal spray composition to provide transmucosal absorption of a therapeutically effective amount of propofol through the oral mucosa of the mammal to the systemic circulatory system of the mammal, the composition comprising: propofol in an amount between about 0.1 and about 99.8 percent by weight of the total composition; and a non-polar solvent in an amount between about 0.05 and about 98.7 percent by weight of the total composition, wherein a therapeutically effective amount of propofol is absorbed through the oral mucosa of the mammal to the mammal's systemic circulatory system.

128. The method of claim 127, further comprising a taste mask and/or flavoring agent in an amount between about 0.01 and about 5 percent by weight of the total composition.

129. The method of claim 128, wherein the propofol is present in an amount between about 1 and about 95 percent by weight of the total composition; the non-polar solvent is present in an amount between about 1 and about 75 percent by weight of the total composition; and the taste mask and/or flavoring agent is present in an amount between about 0.5 and about 4 percent by weight of the total composition.

130. The method of claim 129, wherein the propofol is present in an amount between about 5 and about 90 percent by weight of the total composition; the non-polar solvent is present in an amount between about 5 and about 60 percent by weight of the total composition; and the taste mask and/or flavoring agent is present in an amount between about 1 and about 2 percent by weight of the total composition.

131. The method of claim 127, wherein the solvent comprises a (C₂-C₂₄) fatty acid (C₂-C₆) ester, C₇-C₁₈ hydrocarbon of linear or branched configuration, C₂-C₆ alkanoyl ester, or triglyceride of C₂-C₆ carboxylic acids.

132. The method of claim 131, wherein the solvent comprises a triglyceride of C₂-C₆ carboxylic acids.

133. The method of claim 128, wherein the flavoring agent comprises a synthetic or natural oil of peppermint, oil of spearmint, citrus oil, fruit flavor, sweetener, or mixture thereof.

134. The method of claim 127, further comprising an absorption enhancer in an amount of between about 0.01 to about 5 percent by weight of the total composition.

135. The method of claim 129, further comprising an absorption enhancer in an amount of between about 0.5 to about 4 percent by weight of the total composition.

136. The method of claim 130, further comprising an absorption enhancer in an amount of between about 1 to about 2 percent by weight of the total composition.

137. The method of claim 134, wherein the absorption enhancer comprises oleic acid, 23-lauryl ether, aprotinin, azone, benzalkonium chloride, cetylpyridinium chloride, cetyltrimethylammonium bromide, cyclodextrin, dextran sulfate, lauric acid, lauric acid/propylene glycol, lysophosphatidylcholine, menthol, methoxysalicylate, methyloleate, phosphatidyleholine, polyoxyethylene, polysorbate 80, sodium EDTA (ethylenediamine tetraacetic acid), sodium glycocholate, sodium glycodeoxycholate, sodium lauryl sulfate, sodium salicylate, sodium taurocholate, sodium taurodeoxycholate, sulfoxides or an alkyl glycoside.

138. The method of claim 127, further comprising an antioxidant in an amount of between about 0.01 to about 20 percent by weight of the total composition.

139. The method of claim 129, further comprising an antioxidant in an amount of between about 0.5 to about 10 percent by weight of the total composition.

140. The method of claim 130, further comprising an antioxidant in an amount of between about 1 to about 2 percent by weight of the total composition.

141. The method of claim 138, wherein the antioxidant comprises ascorbyl palmitate, alpha tocopherol, butylated hydroxyanisole or fumaric acid.

142. The method of claim 127, wherein the amount of the spray is predetermined.

143. A method of administering propofol to a mammal, comprising spraying the oral mucosa of the mammal with a propellant free buccal spray composition to provide transmucosal absorption of a therapeutically effective amount of propofol through the oral mucosa of the mammal to the systemic circulatory system of the mammal, the composition comprising: propofol in an amount of between about 0.1 and about 99.8 percent by weight of the total composition; a polar solvent in an amount of between about 0.5 to about 98.7 percent by weight of the total composition; and a non-polar solvent in an amount of between about 0.1 to about 80 percent by weight of the total composition, wherein a therapeutically effective amount of propofol is absorbed through the oral mucosa of the mammal to the mammal's systemic circulatory system.

144. The method of claim 143, wherein the ratio of the polar solvent to the non-polar solvent ranges from about 1:99 to about 99:1.

145. The method of claim 143, further comprising a taste mask and/or flavoring agent in an amount of between about 0.01 and about 5 percent by weight of the total composition.

146. The method of claim 145, wherein the propofol is present in an amount between about 1 to about 95 percent by weight of the total composition, the polar solvent is present in an amount between about 1 to about 75 percent by weight of the total composition, the non-polar solvent is present in an amount between about 0.5 to about 75 percent by weight of the total composition, and the taste mask and/or flavoring agent is present in an amount between about 0.5 to about 4 percent by weight of the total composition.

147. The method of claim 146, wherein the propofol is present in an amount between about 5 to about 90 percent by weight of the total composition, the polar solvent is present in an amount between about 5 to about 60 percent by weight of the total composition, the non-polar solvent is present in an amount between about 1 to about 60 percent by weight of the total composition, and the taste mask and/or flavoring agent is present in an amount between about 1 to about 2 percent by weight of the total composition.

148. The method of claim 143, wherein the polar solvent comprises a polyethylene glycol having a molecular weight between 400 and 1000, C₂ to C₈ mono- and poly-alcohol, or C₇ to C₁₈ alcohol of linear or branched configuration and the non-polar solvent comprises a (C₂-C₂₄) fatty acid (C₂-C₆) ester, C₇-C₁₈ hydrocarbon of linear or branched configuration, C₂-C₆ alkanoyl ester, or triglyceride of C₂-C₆ carboxylic acids.

149. The method of claim 145, wherein the flavoring agent comprises a synthetic or natural oil of peppermint, oil of spearmint, citrus oil, fruit flavor, sweetener, or mixture thereof.

150. The method of claim 143, further comprising an absorption enhancer in an amount of between about 0.01 to about 5 percent by weight of the total composition.

151. The method of claim 146, further comprising an absorption enhancer in an amount of between about 0.5 to about 4 percent by weight of the total composition.

152. The method of claim 147, further comprising an absorption enhancer in an amount of between about 1 to about 2 percent by weight of the total composition.

153. The method of claim 150, wherein the absorption enhancer comprises oleic acid, 23-lauryl ether, aprotinin, azone, benzalkonium chloride, cetylpyridinium chloride, cetyltrimethylammonium bromide, cyclodextrin, dextran sulfate, lauric acid, lauric acid/propylene glycol, lysophosphatidylcholine, menthol, methoxysalicylate, methyloleate, phosphatidylcholine, polyoxyethylene, polysorbate 80, sodium EDTA (ethylenediamine tetraacetic acid), sodium glycocholate, sodium glycodeoxycholate, sodium lauryl sulfate, sodium salicylate, sodium taurocholate, sodium taurodeoxycholate, sulfoxides or an alkyl glycoside.

154. The method of claim 143, further comprising an antioxidant in an amount of between about 0.01 to about 20 percent by weight of the total composition.

155. The method of claim 146, further comprising an antioxidant in an amount of between about 0.5 to about 10 percent by weight of the total composition.

156. The method of claim 147, further comprising an antioxidant in an amount of between about 1 to about 2 percent by weight of the total composition.

157. The method of claim 154, wherein the antioxidant comprises ascorbyl palmitate, alpha tocopherol, butylated hydroxyanisole or fumaric acid.

158. The method of claim 143, wherein the amount of the spray is predetermined.

159. A method of administering propofol to a mammal, comprising spraying the oral mucosa of the mammal with a propellant free buccal spray composition to provide transmucosal absorption of a therapeutically effective amount of propofol through the oral mucosa of the mammal to the systemic circulatory system of the mammal, the composition comprising: propofol in an amount of between about 5 and about 90 percent by weight of the total composition, a polar solvent in an amount between about 0.05 and about 98.7 percent by weight of the total composition, and ascorbyl palmitate in an amount between about 0.01 and about 20 percent by weight of the total composition, wherein a therapeutically effective amount of propofol is absorbed through the oral mucosa of the mammal to the mammal's systemic circulatory system.
